(19) United States
(12) Patent Application Publication
(10) Pub. No.: US 2023/0368914 A1
(43) Pub. Date: Nov. 16, 2023

(54) SYSTEMS AND METHODS FOR COMPUTER-IMPLEMENTED METABOLITE ANALYSIS AND PREDICTION FOR ANIMAL SUBJECTS

(71) Applicant: DSM IP ASSETS B.V., Heerlen (NL)

(72) Inventors: Kevin FREEMAN, Columbia, MD (US); Joshua CLAYPOOL, Columbia, MD (US); Ghislain SCHYNS, Columbia, MD (US); Riccardo SFRISO, Columbia, MD (US)

(21) Appl. No.: 18/022,361

(22) PCT Filed: Aug. 24, 2021
(86) PCT No.: PCT/US2021/047258

§ 371 Date: Feb. 21, 2023

Related U.S. Application Data
(60) Provisional application No. 63/069,584, filed on Aug. 24, 2020.

(51) Int. Cl. G16H 50/20; G16H 50/30; G16H 20/60; A61B 5/00

(52) U.S. Cl. CPC G16H 50/20; G16H 50/30; G16H 20/60; A61B 5/4866

(57) ABSTRACT

In some aspects, the disclosure is directed to methods and systems for identifying a set of predictor metabolites which are predictive of a state of an animal subject. For that purpose, a plurality of data sets of respective ones of a plurality of animal subjects may be obtained, wherein each of the plurality of data sets comprises measurement data comprising an indication of a concentration of each of a plurality of metabolites in a sample of a microbiome of a respective animal subject. A label may be provided at least in part characterizing the state of the animal subject. A feature selection process may be applied to the plurality of data sets to select and thereby identify a subset of the plurality of metabolites of which subset the concentrations are a statistically significant predictor of the state according to the label.

### FIELD OF THE DISCLOSURE

This disclosure generally relates to systems and methods for computer-implemented metabolite analysis and prediction for animal subjects. In particular, this disclosure relates to systems and methods for identifying a set of predictor metabolites which are predictive of a state of an animal subject, such as a health, welfare, or performance state of the animal subject.

### BACKGROUND OF THE DISCLOSURE

Microbiome metabolites are indicative of a health, welfare and/or performance state of animal subjects. These metabolites can have a direct impact on the state of an animal subject, or they can indirectly provide insight into other metabolic processes affecting the animal subject, such as by triggering other processes that directly have an impact on the animal subject's health, welfare and/or performance state or being created as the result of other processes that impact that subject's state. However, the number of metabolites likely to be found in an animal subject is in the tens of thousands, with a similar number of biochemical processes at play, making it difficult to analyze the effects of individual metabolites or make predictions of an animal subject's performance, welfare, or health. Specifically, while microbiome metabolite concentrations can be measured, the high-dimensionality of this data and the complexity of the underlying relationships makes it difficult to extract meaningful insights from such data.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1A is an example heatmap illustrating a concentration of metabolites found in a sample population, in some implementations;

FIG. 1B is a chart of health scores for the sample population of FIG. 1A, according to one implementation;

FIG. 1C is a block diagram illustrating a method for machine learning-based metabolite analysis and prediction, according to some implementations;

FIG. 1D is a graph of relative importance of the metabolites found in the sample population of FIG. 1A, according to some implementations;

FIG. 1E is set of graphs for a subset of metabolites found in the sample population of FIG. 1A and corresponding health scores, according to some implementations;

FIG. 2 is a block diagram of a system for machine learning-based metabolite analysis and prediction, according to some implementations;

FIG. 3 is a flow chart of a method for machine learning-based metabolite analysis and prediction, according to some implementations;

FIGS. 4A and 4B are block diagrams depicting embodiments of computing devices useful in connection with the methods and systems described herein;

FIG. 5 shows a predictive character of a subset of metabolites for a group of animal subjects being treated with a nutritional supplement or not;

FIGS. 6A-6C illustrate the predictive character of a number of metabolites.

The details of various embodiments of the methods and systems are set forth in the accompanying drawings and the description below.

### Definitions

Animal: The term “animal” refers to any animal including humans. Examples of animals are monogastric animals, including but not limited to pigs or swine (including, but not limited to, piglets, growing pigs, and sows); poultry such as turkeys, ducks, quail, guinea fowl, geese, pigeons (including squabs) and chicken (including but not limited to broiler chickens (referred to herein as broilers), chicks, layer hens (referred to herein as layers)); pets such as cats and dogs; horses; crustaceans (including but not limited to shrimps and prawns) and fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish). Preferably, in all embodiments of the present invention, the animal is a mammal or a non-human animal incl. a fish or a bird.

The mammal of this invention can be any species. Preferred mammals according to this invention are humans, swine, bovines, equines, canines, felines, rabbits, and bovines.

Animal feed: The term “animal feed” refers to any compound, preparation, or mixture suitable for, or intended for intake by an animal. Animal feed for a monogastric animal typically comprises concentrates as well as vitamins, minerals, enzymes, eubiotics, prebiotics, probiotics (as for example direct fed microbials), amino acids and/or other feed additives (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Concentrates: The term “concentrates” means feed with high protein and energy concentrations, such as fish meal, molasses, oligosaccharides, sorghum, seeds and grains (either whole or prepared by crushing, milling, etc. from e.g. corn, oats, rye, barley, wheat), oilseed press cake (e.g. from cottonseed, safflower, sunflower, soybean (such as soybean meal), rapeseed/canola, peanut or groundnut), palm kernel cake, yeast derived material and distillers grains (such as wet distillers grains (WDS) and dried distillers grains with solubles (DDGS)).

Feed additives: Feed additives are vitamins, minerals, enzymes, eubiotics, prebiotics, probiotics (as for example direct fed microbials), amino acids. The incorporation of the feed additives (feed supplement compositions) is in practice carried out using a premix. A premix designates a preferably uniform mixture of one or more micro-ingredients with diluent and/or carrier. Premixes are used to facilitate uniform dispersion of micro-ingredients in a larger mix. A premix can be added to feed ingredients or to the drinking water as solids (for example as water soluble powder) or liquids.

Enzymes are used preliminary for improving feed utilization and digestibility of feed. Examples are phytases, proteases, carbohydrases and mixtures thereof. Another new category of feed enzymes are “gut health” enzymes as for example muramidases which have a positive influence on the gut micro flora and animal health and welfare.

Enzymes can be classified on the basis of the handbook Enzyme Nomenclature from NC-IUBMB, 1992), see also the ENZYME site at the internet: http://www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB), Academic Press, Inc., 1992, and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

A feed enzyme composition is selected from the group comprising of acetylxylan esterase (EC 3.1.1.23), acylglycerol lipase (EC 3.1.1.72), alpha-amylase (EC 3.2.1.1), beta-amylase (EC 3.2.1.2), arabinofuranosidase (EC 3.2.1.55), cellobiohydrolases (EC 3.2.1.91), cellulase (EC 3.2.1.4), feruloyl esterase (EC 3.1.1.73), galactanase (EC 3.2.1.89), alpha-galactosidase (EC 3.2.1.22), beta-galactosidase (EC 3.2.1.23), beta-glucanase (EC 3.2.1.6), beta-glucosidase (EC 3.2.1.21), triacylglycerol lipase (EC 3.1.1.3), lysophospholipase (EC 3.1.1.5), lysozyme (EC 3.2.1.17), alpha-mannosidase (EC 3.2.1.24), beta-mannosidase (mannanase) (EC 3.2.1.25), phytase (EC 3.1.3.8, EC 3.1.3.26, EC 3.1.3.72), phospholipase A1 (EC 3.1.1.32), phospholipase A2 (EC 3.1.1.4), phospholipase D (EC 3.1.4.4), protease (EC 3.4), pullulanase (EC 3.2.1.41), pectinesterase (EC 3.1.1.11), xylanase (EC 3.2.1.8, EC 3.2.1.136), beta-xylosidase (EC 3.2.1.37), or any combination thereof.

Eubiotics are compounds which are designed to give a healthy balance of the micro-flora in the gastrointestinal tract. Eubiotics cover a number of different feed additives, such as probiotics, prebiotics, phytogenics (essential oils) and organic acids which are described in more detail below.

Prebiotics: Prebiotics are substances that induce the growth or activity of microorganisms (e.g., bacteria and fungi) that contribute to the well-being of their host. Prebiotics are typically non-digestible fiber compounds that pass undigested through the upper part of the gastrointestinal tract and stimulate the growth or activity of advantageous bacteria that colonize the large bowel by acting as substrate for them. Normally, prebiotics increase the number or activity of bifidobacteria and lactic acid bacteria in the GI tract.

Yeast derivatives (inactivated whole yeasts or yeast cell walls) can also be considered as prebiotics. They often comprise mannan-oligosaccharids, yeast beta-glucans or protein contents and are normally derived from the cell wall of the yeast, Saccharomyces cerevisiae.

Organic Acids: Organic acids (C1-C7) are widely distributed in nature as normal constituents of plants or animal tissues. They are also formed through microbial fermentation of carbohydrates mainly in the large intestine. They are often used in swine and poultry production as a replacement of antibiotic growth promoters since they have a preventive effect on the intestinal problems like necrotic enteritis in chickens and Escherichia coli infection in young pigs. Organic acids can be sold as mono component or mixtures of typically 2 or 3 different organic acids. Examples of organic acids are short chain fatty acids (e.g. formic acid, acetic acid, propionic acid, butyric acid), medium chain fatty acids (e.g. caproic acid, caprylic acid, capric acid, lauric acid), di/tri-carboxylic acids (e.g. fumaric acid), hydroxy acids (e.g. lactic acid), aromatic acids (e.g. benzoic acid), citric acid, sorbic acid, malic acid, and tartaric acid or their salt (typically sodium or potassium salt such as potassium diformate or sodium butyrate).

Amino Acids: The composition or the animal feed of the invention may further comprise one or more amino acids. Examples of amino acids which are used are lysine, alanine, beta-alanine, threonine, methionine and tryptophan.

Vitamins: The composition or the animal feed may include one or more vitamins, such as one or more fat-soluble vitamins and/or one or more water-soluble vitamins. In addition, the composition or the animal feed may optionally include one or more minerals, such as one or more trace minerals and/or one or more macro minerals.

Usually fat and water soluble vitamins, as well as trace minerals form part of a so-called premix intended for addition to the feed, whereas macro minerals are usually separately added to the feed.

Non-limiting examples of fat soluble vitamins include vitamin A, vitamin D3, vitamin E, and vitamin K, e.g., vitamin K3.

Non-limiting examples of water soluble vitamins include vitamin C, vitamin B12, biotin and choline, vitamin B1, vitamin B2, vitamin B6, niacin, folic acid and panthothenate, e.g., Ca-D-panthothenate.

Minerals: Non-limiting examples of trace minerals include boron, cobalt, chloride, chromium, copper, fluoride, iodine, iron, manganese, molybdenum, iodine, selenium and zinc.

Non-limiting examples of macro minerals include calcium, magnesium, phosphorus, potassium and sodium.

Other feed ingredients: The composition or the animal feed of the invention may further comprise colouring agents, stabilisers, growth improving additives and aroma compounds/flavourings, polyunsaturated fatty acids (PUFAs); reactive oxygen generating species, antioxidants, anti-microbial peptides, anti-fungal polypeptides and mycotoxin management compounds.

Examples of colouring agents are carotenoids such as beta-carotene, astaxanthin, and lutein.

Examples of aroma compounds/flavourings are creosol, anethol, deca-, undeca- and/or dodeca-lactones, ionones, irone, gingerol, piperidine, propylidene phatalide, butylidene phatalide, capsaicin and tannin.

Examples of antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

Examples of polyunsaturated fatty acids are C18, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Antioxidants can be used to limit the number of reactive oxygen species which can be generated such that the level of reactive oxygen species is in balance with antioxidants.

Mycotoxins, such as deoxynivalenol, aflatoxin, zearalenone and fumonisin can be found in animal feed and can result in negative animal performance or illness. Compounds which can manage the levels of mycotoxin, such as via deactivation of the mycotoxin or via binding of the mycotoxin, can be added to the feed to ameliorate these negative effects.

Feed Conversion Ratio (FCR): FCR is a measure of an animal's efficiency in converting feed mass into increases of the desired output. Animals raised for meat—such as swine, poultry and fish—the output is the mass gained by the animal. Specifically FCR is calculated as feed intake divided by weight gain, all over a specified period. Improvement in FCR means reduction of the FCR value. A FCR improvement of 2% means that the FCR was reduced by 2%.

Feed Premix: The incorporation of the composition of feed additives as exemplified herein above to animal feeds, for example poultry feeds, is in practice carried out using a concentrate or a premix. A premix designates a preferably uniform mixture of one or more microingredients with diluent and/or carrier. Premixes are used to facilitate uniform dispersion of micro-ingredients in a larger mix. A premix according to the invention can be added to feed ingredients or to the drinking water as solids (for example as water soluble powder) or liquids.

Nutrient: The term “nutrient” in the present invention means components or elements contained in dietary feed for an animal, including water-soluble ingredients, fat-soluble ingredients and others. The example of water-soluble ingredients includes but is not limited to carbohydrates such as saccharides including glucose, fructose, galactose and starch; minerals such as calcium, magnesium, zinc, phosphorus, potassium, sodium and sulfur; nitrogen source such as amino acids and proteins, vitamins such as vitamin B1, vitamin B2, vitamin B3, vitamin B6, folic acid, vitamin B12, biotin and phatothenic acid. The example of the fat-soluble ingredients includes but is not limited to fats such as fat acids including saturated fatty acids (SFA); mono-unsaturated fatty acids (MUFA) and poly-unsaturated fatty acids (PUFA), fibre, vitamins such as vitamin A, vitamin E and vitamin K. A nutrient may be supplied as a nutritional additive to an animal subject, for example via feed or drinking water of the animal subject.

Biomarkers and Metabolites

Metabolite: The term “metabolites” in the present invention may refer to any substance involved in a mammal's metabolism. Such metabolites may be the immediate by-product of a metabolic process. Typically, metabolites are biomolecules which are smaller in size than proteins and nucleic acids and other large biomolecules. Although mostly naturally occurring, metabolites can be produced artificially for industrial and pharmaceutical uses. The metabolites are often grouped into two major types: primary and secondary. While primary metabolites are those that are directly involved in the growth, development, and reproduction of an organism, secondary metabolites are those that are not or more indirectly. Examples of secondary metabolites include, but are not limited to, antimicrobials, anti-inflammatory molecules, hormones and neuromodulators. The term ‘metabolites’ may include both primary and secondary metabolites. Such metabolites may be categorized by super pathway and sub pathway. When measuring metabolites in a sample of a microbiome, any suitable number may be measured, e.g., several tens or hundreds, e.g., around or above 25, 50, 75, 100, 150, 200, 250, 300, 400, 500, 600, 700, 800, 1000, 1200, 1500, etc. The size of the set of predictor metabolites may be a subset of this set, of which the number, i.e., the size of the subset, may be a user-configurable parameter or may be automatically determined by the feature selection algorithm, e.g., to have a prediction accuracy above a desired value. For example, the subset may have a size of around or below 1%, 2%, 3%, 4%, 5%, 8%, 10%, 12%, 15%, 20% etc. of the set of originally measured metabolites.

A biomarker, or biological marker is a measurable indicator of some biological state or condition. Biomarkers are often measured and evaluated using blood, urine, or soft tissues to examine normal biological processes, pathogenic processes, or pharmacologic responses to a therapeutic intervention. Relevant biomarkers in animal health which provide insights on different health challenges include but are not limited to: Albumin, Anion Gap, AST, Calcium, Carotenoids, Chloride, Creatine Kinase, Globulin, Glucose, Hematocrit, Hemoglobin, Ionized Calcium, Phosphorus, Potassium, Sodium, Total Carbon Dioxide, Total Protein, Uric Acid. Biomarkers are examples of metabolites. As such, the term ‘metabolite’ as used in this specification includes biomarkers, including but not limited the above-mentioned biomarkers.

### DETAILED DESCRIPTION

For purposes of reading the description of the various embodiments below, the following descriptions of the sections of the specification and their respective contents may be helpful:
Section A describes embodiments of systems and methods for computer-implemented metabolite analysis and prediction; and
Section B describes a computing environment which may be useful for practicing embodiments described herein.

### A. Systems and Methods for Computer-Implemented Metabolite Analysis and Prediction

Metabolites are molecules that are the result or intermediate product of a metabolic process, including molecules used for signaling or triggers for other processes, molecules that provide fuel for other processes, etc. Examples include proteins, lipids, carbohydrates, steroids, antibiotics, phenolics, and other molecules. Microbiome metabolites, such as those found in the gastrointestinal tract, respiratory tract, oral cavity, skin, or blood, are indicative of health, welfare and performance, e.g., in terms of growth, of animal subjects. These metabolites can have a direct impact on the health or welfare or performance of an animal subject, or they can indirectly provide insight into other metabolic processes affecting the animal subject, such as by triggering other processes that directly have an impact on the animal subject's health or being created as the result of other processes that impact that subject's health. However, the number of metabolites likely to be found in an animal subject is in the tens of thousands, with a similar number of biochemical processes at play, making it difficult to analyze the effects of individual metabolites or make predictions of an animal subject's growth or health. Specifically, while microbiome metabolite concentrations can be measured, the high-dimensionality of this data and the complexity of the underlying relationships makes it difficult to extract insights.

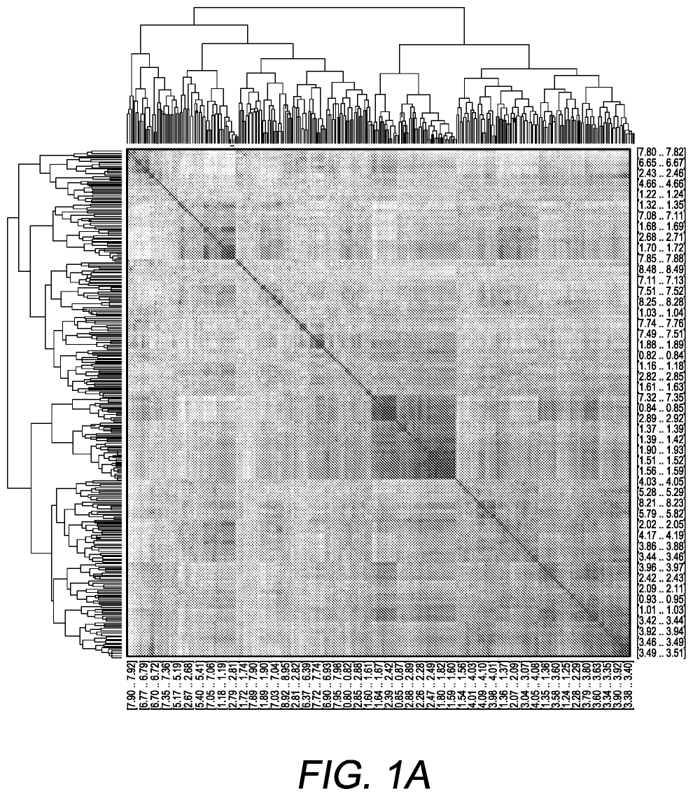

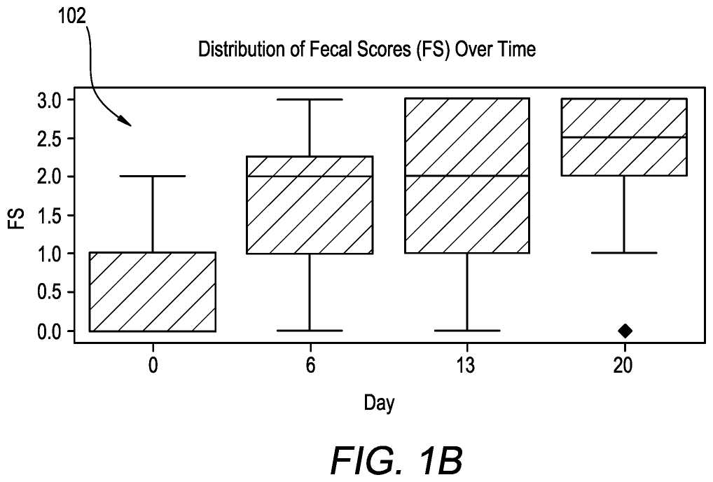

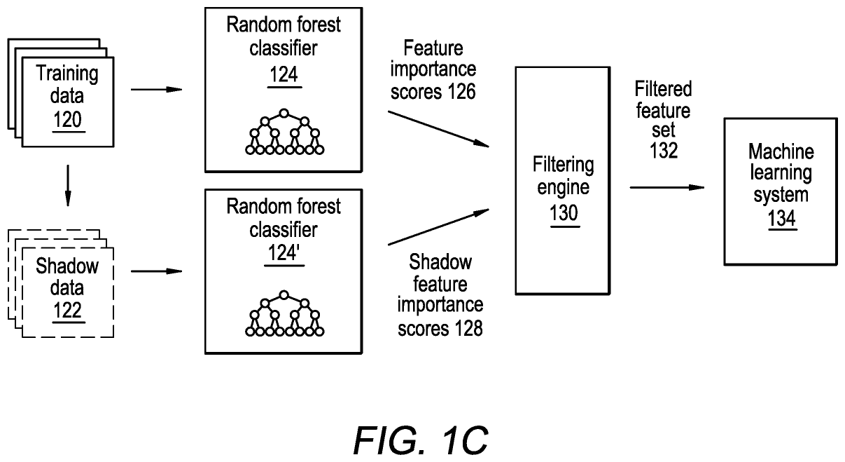

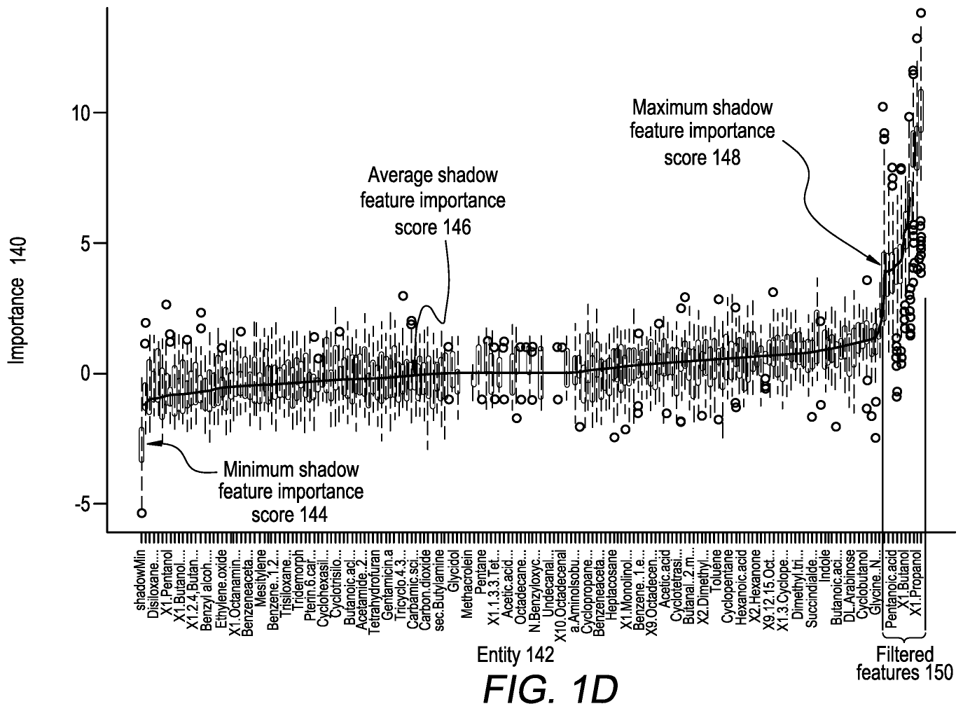

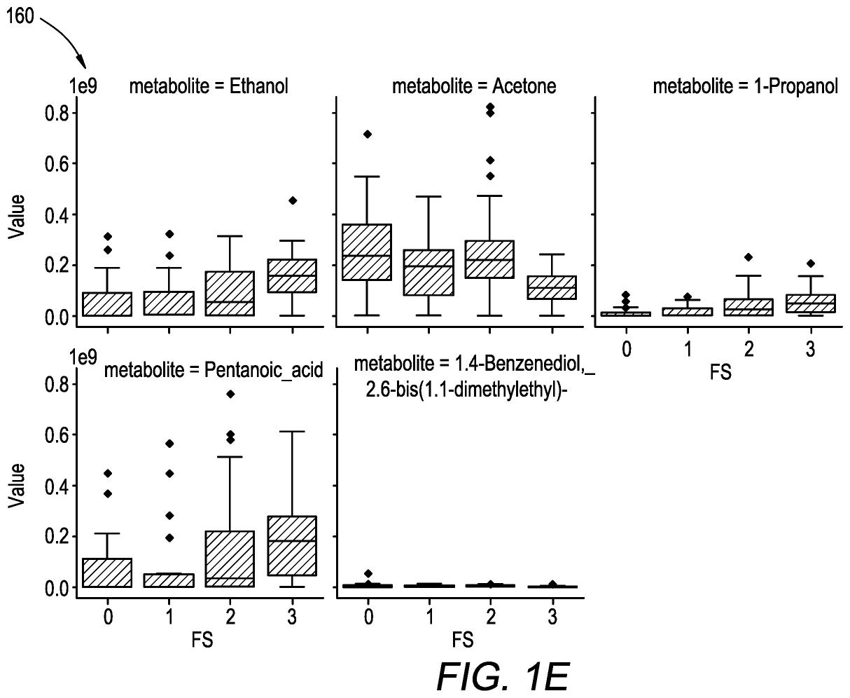

For example, FIG. 1A is an example heatmap 100 illustrating a concentration of metabolites found in a sample population of 40 swine during one longitudinal study investigating metabolic causes and early warning signs of diarrhea in piglets. The swine were all fed a high protein diet and monitored over the course of 3 weeks. The metabolites were measured from fecal samples using time-of-flight (TOF) mass spectrometry. This TOF mass spectrometry identified 291 distinct molecules (metabolites) in the swine fecal samples, and concentrations were measured. As shown, the resulting data is highly complex and cluster analysis (as shown in the dendrogram) may provide few if any insights.

Metabolomics data and fecal scores (FS) from the pigs were collected at 4 timepoints throughout the study. Fecal scoring is a subjective scoring done by a human by examining the feces. In some implementations, a fecal score can be 0, 1, 2, or 3, where a score of 0 indicates no signs of diarrhea and a score of 3 indicates severe diarrhea. FIG. 1B is a chart of health scores for the sample population of FIG. 1A, according to one implementation. During the longitudinal study, it was found that average FS increased over time in the study. Predicting this result from the 291 metabolites may be difficult, if not impossible, due to the complexity and scale of the data. However, a small subset of the metabolites may be predictive of fecal scoring (and/or other health or welfare or performance metrics, such as growth rates, mortality, body fat percentage, etc.), and can be identified via implementations of the systems and methods discussed herein. This subset of metabolites can then be monitored as an early warning sign of declining gut health in swine. Furthermore, the systems and methods discussed herein provide an efficient analytic tool to identify a subset of significant metabolites that are predictive of health, welfare, or performance within any type of sample or subject, including blood, oral, or skin samples, in animals. For example, in various implementations, the systems and methods discussed herein may enable prediction of various aspects of animal health or welfare or performance, including growth rates, body weight gain, water and feed consumption, feed conversion ratios (e.g., feed weight to body weight gain), lean muscle mass, weaning weights and ages, egg production, fertility (e.g. number of offspring per litter, numbers of litters per year, numbers of offspring per year, etc.), mortality (including infant mortality as well as overall mortality rates), muscular endurance and athletic capability, methane emission rates, resting heart rates, and other such metrics, including standardized scoring metrics based on subjective or objective observation of phenotypes or data and translated or scaled into a scoring range (e.g. fecal quality scores, hair shedding scores, cattle foot scores, pulmonary arterial pressure scores, marbling scores, etc.), or any other quantizable trait or characteristic that is influenced by metabolites, referred to generally herein as health and welfare and performance scores or metrics.

To identify significant metabolites, in some embodiments, a multi-stage machine learning system may be utilized with training data and shadow training data generated from randomization or shuffling of training data. FIG. 1C is a block diagram illustrating a method for machine learning-based metabolite analysis and prediction, according to some implementations. Training data 120, which may comprise measurements of metabolites in samples from a plurality of subjects and corresponding health or performance metrics of the animal subjects may be classified via a feature selection process, such as a random forest classifier 124 or similar classifier, to generate importance or significance values or feature importance scores 126 for each metabolite representing the corresponding metabolite's relative correlation with or contribution to a specific outcome or metric. These scores may be distributed over a wide or narrow range, and with a large number of metabolites, it may be difficult to determine which metabolites are particularly predictive. In some implementations of systems not utilizing the methods discussed herein, a number n of features having the highest corresponding importance scores may be utilized for subsequent data analysis and prediction, but this number may be selected arbitrarily and may be over-inclusive (requiring additional computing resources for subsequent analysis, decreasing efficiency, and increasing processing time required) or may be under-inclusive (decreasing predictive accuracy).

To address this deficiency and select an optimized subset of metabolites for analysis, a second set of “shadow” training data 122 may be generated by randomizing or shuffling the training data 120 (e.g. for each sample, assigning a random measurement value to each metabolite in some implementations; or by randomly shuffling or swapping measurement values for one or more samples without changing corresponding health or performance metric measurements in some implementations, such as replacing measurements a₁, a₂, . . . , aₙ for a sample s₁ with performance metric p₁, with measurements b₁, b₂, . . . , bₙ from a second sample s₂ with performance metric p₂). The resulting shadow training data 122 thus comprises false data that lacks genuine predictive ability for the health or performance metric (e.g. measurements b₁, b₂, . . . , bₙ with performance metric p₁, which did not occur in reality). The shadow training data 122 may be similarly analyzed by a classifier, such as a random forest classifier 124′, to create a set of shadow feature importance scores 128.

A filtering engine 130 may select a subset of features or metabolites (filtered feature set 132) from the training data by selecting only those metabolites whose feature importance scores 126 exceed the shadow feature importance scores 128 (e.g. greater than a maximum shadow feature importance score or greater than an average shadow feature importance score, in various implementations). For example, FIG. 1D is a graph of relative importance scores 140 of the metabolites (entities 142) of the training data and shadow training data for the sample population of FIG. 1A, according to some implementations. The minimum 144, average 146, and maximum 148 importance scores determined via the shadow training data are shown, and a subset of importance scores from the training data exceed the maximum importance scores (corresponding to filtered features 150).

These filtered features 150 may have the highest relevancy to the health or performance metrics, and may represent an optimized set of features for subsequent analysis by a machine learning system. FIG. 1E is set of graphs 160 for a filtered subset of metabolites found in the sample population of FIG. 1A and corresponding fecal scores, according to some implementations. By processing the longitudinal study data as discussed above, 5 metabolites were identified that provide optimized predictive power for the dataset: ethanol, acetone, 1-propanol, pentanoic acid, and 1,4-Benzenediol, 2,6-bis(1,1-dimethylethyl)-.

Returning to FIG. 1C, the resulting filtered feature set 132 or subset of features may in some embodiments be used to train a machine learning system 134, such as a neural network or other predictive engine. For example, the training data 120 may be filtered to the subset of metabolites selected by the filtering engine 130 and a neural network may be trained with the subset of metabolite sample measurements in the training data and the corresponding health or performance metrics. Such a machine learning system may be used in various applications, for example in monitoring or prediction. For example, the 5 metabolites identified above can be monitored for changes as an early warning system for gut health in swine.

As discussed above, the system is not limited to gut health or swine, but may be utilized with samples of any microbiome including those of the gastrointestinal tract, respiratory tract, oral cavity, skin or blood of animals, and with health scores, performance scores, growth rates, or any other such metrics. For example, implementations of the systems and methods discussed herein may be utilized with samples of a skin microbiome and subjective observations of skin changes or reactions to a stimulus (e.g. radiation induced skin injury) against a standardized score (e.g. from 1.0 corresponding to no effect, to 2.5 corresponding to marked erythema or dry desquamation, to 5.5 corresponding to necrosis), which may allow for analysis of metabolites involved in skin damage or healing and prediction of or early diagnosis of conditions. Similarly, these systems and methods may be applied to any microbiome samples of metabolites of animal subjects with corresponding quantifiable subjective, semi-subjective, or objective metrics.

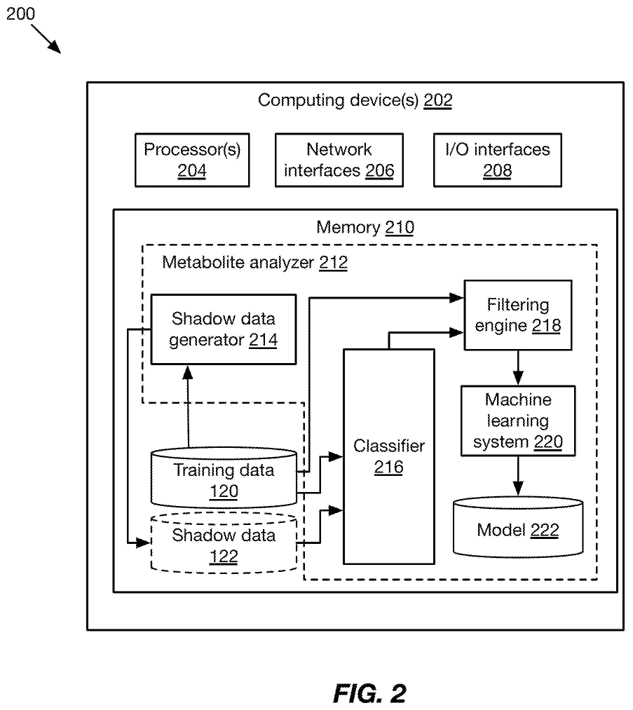

FIG. 2 is a block diagram of a system 200 for machine learning-based metabolite analysis and prediction, according to some implementations. The system includes one or more computing devices 202, which may comprise servers, workstations, desktop computers, laptop computers, rackmount computers, appliances, clusters of computers, virtual computing devices executed by one or more physical computing devices (e.g. a cloud of servers), or any other combination of these or other computing devices. The computing devices may comprise one or more processors 204 (which may include physical and/or virtual processors), one or more network interfaces 206 (e.g. for communicating with other computing devices 202 over a network, such as when deployed in a cluster or for retrieval of data such as training data 120 stored on a data server or other storage device), and one or more input/output interfaces 208 for interaction with the system. In some implementations, processors 204 may include one or more co-processors optimized for machine learning, such as tensor processing units (TPUs) or other application specific integrated circuits (ASICs) configured for executing a machine learning or classifier algorithm. Computing device 202 may include or communicate with one or more memory devices 210, including internal storage, external storage, network storage, or other such devices, including virtual storage devices provided by a storage server or network.

Computing device 202 may execute a metabolite analyzer 212, which may comprise an application, server, service, daemon, routine, or other executable logic for analyzing and filtering training data to select subsets of metabolites for training a machine learning system. In some implementations, metabolite analyzer 212 may also process new sample data via the trained machine learning system. Metabolite analyzer 212 may receive training data 120, which may be in any suitable format, such as an array, database, spreadsheet, string of comma-separated values, multi-dimensional vector, or other such format or data structure. Training data 120 may comprise measurements of concentrations of metabolites from a sample of a microbiome, such as intestinal or fecal samples, blood samples, saliva samples, skin samples or any other type and form of samples, and may also comprise one or more scores associated with the sample such as a health score, growth score, fecal score, performance score, or other such metric.

Metabolite analyzer 212 may include a shadow data generator 214. Shadow data generator 214 may comprise an application, server, service, daemon, routine, or other executable logic for generating shadow training data 122 from training data 120. As discussed above, in some implementations, shadow training data 122 may be generated by randomly shuffling or swapping metabolite concentration or measurement values for different samples while maintaining the associated health or performance score or metric, such that the measurement values no longer correspond with the original score or metric. In a similar implementation, shadow training data 122 may be generated by randomly shuffling health or performance scores or metrics for samples while maintaining measurement data for each sample. In other implementations, shadow training data 122 may be generated by creating random metabolite concentration or measurement values and/or scores or metrics (e.g. creating new “fake” or shadow samples for the shadow training set). In some implementations, a mix of shuffled and randomly generated data may be utilized.

Metabolite analyzer 212 may include a classifier 216 or communicate with another application or computing device 202 executing a classifier 216. Classifier 216 may comprise an application, server, service, daemon, routine, or other executable logic for applying a classification algorithm to a data set, such as an ensemble algorithm including a random forest classifier or Bayesian classifier; a kernel method such as a support vector machine or principal component analyzer; or any other type and form of classifier for identifying variable importance or feature scores. Classifier 216 may generate importance scores for each metabolite or entity in the training data 120 and shadow training data 122.

Metabolite analyzer 212 may comprise a filtering engine 218. Filtering engine 218 may comprise an application, server, service, daemon, routine, or other executable logic for selecting metabolites or entities in the training data 120 based on a comparison of their importance scores to importance scores for metabolites or entities in the shadow training data 122. For example, in some implementations, filtering engine 218 may select a subset of one or more metabolites or entities in the training data 120 having importance scores that are higher than a highest or maximum importance score found from the shadow training data 122. In other implementations, filtering engine 218 may select a subset of one or more metabolites or entities in the training data 120 having importance scores that are higher than an average importance score found from the shadow training data 122. In some implementations, a dynamic threshold may be utilized based on the importance scores of the shadow training data (e.g. a threshold equal to two standard deviations above the average, a threshold equal to 95% of the maximum value, etc.). The threshold may be tuned optimize the subset selection for under and over-inclusiveness.

In some implementations, the filtered data set may be utilized with a metabolic network to identify additional metabolites and/or enzymes that may be relevant to the health or performance score or metric. For example, given a metabolic network comprising metabolite nodes and enzyme edges that convert from one metabolite to another, metabolites from the filtered data set may be identified in the network and neighboring (e.g. upstream or downstream) metabolites and/or enzymes may be identified for sampling (e.g. adding to the filtered data set as having likely predictive qualities or sampled for a subsequent training iteration).

In some implementations, metabolite analyzer 212 may comprise a machine learning system 220 or communicate with a machine learning system 220 executed by another computing device 202. Machine learning system 220 may comprise an application, server, service, daemon, routine, or other executable logic for performing a supervised learning algorithm utilizing a subset of metabolite sample measurements and health or performance scores of training data 120. Machine learning system 220 may comprise a random forest classifier, a support vector machine, a neural network, or any other type and form or combination of classification or machine learning algorithms. Utilizing the filtered training data, the machine learning system 220 may generate a model 222 of hyperparameters, weights, and/or coefficients for executing the machine learning system on new sample data to predict performance or health scores or metrics.

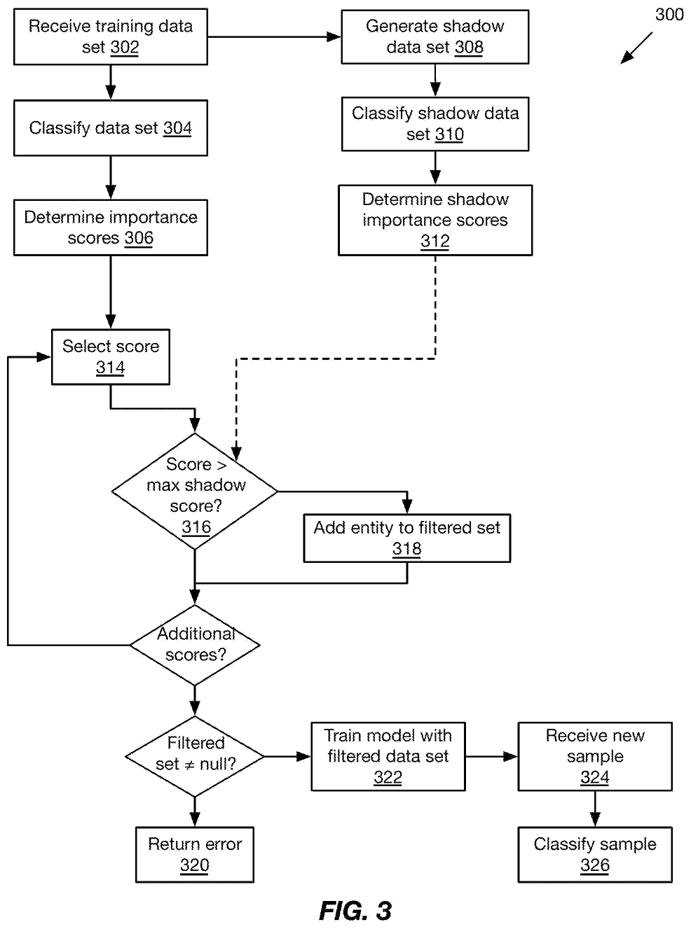

FIG. 3 is a flow chart of a method 300 for machine learning-based metabolite analysis and prediction, according to some implementations. At step 302, a computing device may receive an initial or training data set. The initial data set may comprise a plurality of identifications or measurements of a concentration of each of a plurality of metabolites in a corresponding plurality of samples, such as oral, lung, gut, skin or blood samples from one or more animal subjects, and a score or metric associated with each sample, such as a health score or performance score or metric as discussed above (e.g. fecal score, weight gain score, body fat percentage, etc.). The concentrations may be measured through chromatography, in some implementations, such as gas or liquid chromatography.

At step 304, the initial data set may be classified. The classification may comprise executing a random forest algorithm, in some implementations. In some implementations, the input data (e.g. measurements and/or scores) may be pre-processed for classification, including normalizing or scaling the measurements and/or scores to a predetermine range or ranges, re-ordering the data, filtering incomplete data, or otherwise preparing the data for classification.

At step 306, importance scores calculated for each metabolite (e.g. importance of the metabolite for contributing to a corresponding health or performance metric). For example, in some implementations using a random forest classifier, importance scores may be calculated as a comparison of prediction accuracy between samples and out-of-bag samples or Gini impurity.

Subsequently or simultaneously in parallel, in various implementations, a shadow data set may be generated from the initial data set. The shadow data set may be generated from a random reshuffling of metabolite measurements and/or health or performance scores or metrics between samples in the initial data set, in some implementations, or may be generated with new random values in other implementations. At step 310, the shadow data set may be classified, and at step 312, importance scores may be determined for the metabolites of the shadow data set, similar to steps 304 and 306 respectively.

Importance scores from the initial data set may be compared to the importance scores from the shadow set to identify scores above a threshold and filter out a subset of metabolites. For example, at step 314 in some implementations, an importance score for a metabolite of the initial data set may be selected, and at step 316, the score may be compared to the importance scores for the metabolites of the shadow data set. If the importance score for the metabolite of the initial data set is greater than a threshold, such as a maximum importance score of the shadow data set, then at step 318, the metabolite may be added to the filtered data set; otherwise, the metabolite may be excluded from the filtered data set. Steps 314-318 may be repeated iteratively for each metabolite in the initial data set. As discussed above, in some implementations, the filtered data set may be enhanced or expanded with one or more additional metabolites or enzymes extracted from a metabolic network or graph (e.g. metabolites or enzymes neighboring selected or filtered metabolites).

If the filtered data set is empty (e.g. no importance score of the initial data set exceeds the threshold or maximum importance score of the shadow data set), then at step 320, the computing device may return an error (e.g. indicating additional training data is needed). Otherwise, at step 322 in some implementations, a machine learning system may be used to train a model based on the filtered initial data set (e.g. the health or performance scores or metrics for each sample and the subset of the metabolite measurements corresponding to the filtered or selected metabolites) to predict health or performance scores or metrics associated with samples.

In some implementations, at step 324, a new metabolite sample for an animal subject may be received, and at step 326, the sample may be classified according to the trained machine learning model to predict a corresponding health or performance score or metric. In some implementations, based on the predicted health or performance score or metric, further actions may be performed for the animal subject, such a remediation or preventative action. For example, in some implementations, responsive to a score or metric below a threshold, an additive or supplement (e.g. antibiotics, vitamins, etc.) may be provided to feed for the animal subject, e.g. by an automated feeding system under direction of the computing device. In other implementations, responsive to the predicted score or metric being below the threshold, the animal subject may be quarantined from other subjects (e.g. by an automatic sorting system or gate under control of the computing device).

Accordingly, the systems and methods discussed herein provide a multi-stage machine learning system that provides more efficient prediction of health or performance scores or metrics at reduced computational cost relative to analysis of a full set of metabolic data.

In one aspect, the present disclosure is directed to a method for pre-processing metabolite data for machine learning-based analysis. The method includes receiving, by a computing device, a plurality of initial data sets, each data set comprising an identification of a concentration of each of a plurality of metabolites in a sample and a score or metric associated with the sample. The method also includes creating, by the computing device, a corresponding plurality of additional data sets, each additional data set comprising the score from a corresponding initial data set and a random resorting of the concentrations of each of the plurality of metabolites from the corresponding initial data set. The method also includes generating, by the computing device via a first classifier using the plurality of additional data sets, a first importance score for each of the plurality of metabolites. The method also includes identifying, by the computing device, a maximum first importance score of the plurality of metabolites generated using the plurality of additional data sets. The method also includes generating, by the computing device via the first classifier using the plurality of initial data sets, a second importance score for each of the plurality of metabolites. The method also includes selecting, by the computing device, a subset of the plurality of metabolites with second importance scores exceeding the maximum first importance score. The method also includes filtering, by the computing device, the identifications of the concentrations of each of the plurality of metabolites of the plurality of initial data sets according to the selected subset of the plurality of metabolites. In some implementations, the method includes training, by the computing device using the filtered plurality of initial data sets, a machine learning system to predict scores or metrics associated with samples.

In some implementations, each sample comprises a sample of metabolites derived from both the animal subject and the microbiome, and the score associated with the sample comprises a health score of the animal subject. In a further implementation, the microbiome of the animal subject is sampled from the gastrointestinal tract and the health score is a fecal score or animal performance score. In another further implementation, the microbiome of the animal subject is sampled from the animal subject's blood and the health score is an animal performance score. In another further implementation, the method includes predicting, by the computing device using the trained machine learning system, a health score above a threshold for a new sample; and providing a control signal, by the computing device to an automated feeding system responsive to the predicted health score being above the threshold, to modify a supplement concentration for the animal subject.

In some implementations, the method includes filtering the identifications of the concentrations of each of the plurality of metabolites of the plurality of initial data sets by removing, from the initial data set, identifications of metabolites associated with second importance scores that are equal to or less than the maximum first importance score. In some implementations, the machine learning system comprises a neural network; and wherein training the neural network further comprises providing the filtered plurality of initial data sets to the neural network in a supervised learning process. In some implementations, the method includes identifying, within a metabolic network comprising nodes corresponding to metabolites and edges corresponding to enzymes converting between metabolites, one or more metabolites connected via an edge to at least one metabolite of the selected subset of the plurality of metabolites with second importance scores exceeding the maximum first importance score. In a further implementation, the method includes recording, to a data structure stored in a memory of the computing device, the identified one or more metabolites.

In another aspect, the present disclosure is directed to a system for pre-processing metabolite data for machine learning-based analysis. The system includes a computing device comprising a processor executing a first classifier and a machine learning engine. The processor is configured to: receive a plurality of initial data sets, each data set comprising an identification of a concentration of each of a plurality of metabolites in a sample and a score or metric associated with the sample; create a corresponding plurality of additional data sets, each additional data set comprising the score or metric from a corresponding initial data set and a random resorting of the concentrations of each of the plurality of metabolites from the corresponding initial data set; generate, via the first classifier using the plurality of additional data sets, a first importance score for each of the plurality of metabolites; identify a maximum first importance score of the plurality of metabolites generated using the plurality of additional data sets; generate, via the first classifier using the plurality of initial data sets, a second importance score for each of the plurality of metabolites; select a subset of the plurality of metabolites with second importance scores exceeding the maximum first importance score; filter the identifications of the concentrations of each of the plurality of metabolites of the plurality of initial data sets according to the selected subset of the plurality of metabolites; and train, using the filtered plurality of initial data sets, the machine learning system to predict scores or metrics associated with samples.

In some implementations, each sample comprises a sample of metabolites derived from both the animal subject and the microbiome, and the score or metric associated with the sample comprises a health score of the animal subject. In a further implementation, the microbiome of the animal subject is a fecal sample and the health score is a fecal score. In a still further implementation, the processor is further configured to: predict, using the trained machine learning system, a fecal score above a threshold for a new fecal sample; and provide a control signal, to an automated feeding system responsive to the predicted fecal score being above the threshold, to modify a supplement concentration for the animal subject.

In some implementations, the processor is further configured to remove, from the initial data set, identifications of metabolites associated with second importance scores that are equal to or less than the maximum first importance score. In some implementations, the machine learning system comprises a neural network. In a further implementation, the processor is further configured to provide the filtered plurality of initial data sets to the neural network in a supervised learning process. In some implementations, the processor is further configured to identify, within a metabolic network comprising nodes corresponding to metabolites and edges corresponding to enzymes converting between metabolites, one or more metabolites connected via an edge to at least one metabolite of the selected subset of the plurality of metabolites with second importance scores exceeding the maximum first importance score. In a further implementation, the computing device further comprises a memory, and the processor is further configured to record, to a data structure stored in the memory, the identified one or more metabolites.

In another aspect, the present disclosure is directed to a non-transitory computer readable medium comprising one or more instructions, the execution of which cause a processor of a computing device to: receive a plurality of initial data sets, each data set comprising an identification of a concentration of each of a plurality of metabolites in a sample and a score or metric associated with the sample; create a corresponding plurality of additional data sets, each additional data set comprising the score or metric from a corresponding initial data set and a random resorting of the concentrations of each of the plurality of metabolites from the corresponding initial data set; generate, via a first classifier using the plurality of additional data sets, a first importance score for each of the plurality of metabolites; identify a maximum first importance score of the plurality of metabolites generated using the plurality of additional data sets; generate, via the first classifier using the plurality of initial data sets, a second importance score for each of the plurality of metabolites; select a subset of the plurality of metabolites with second importance scores exceeding the maximum first importance score; filter the identifications of the concentrations of each of the plurality of metabolites of the plurality of initial data sets according to the selected subset of the plurality of metabolites; and train, using the filtered plurality of initial data sets, a machine learning system to predict scores or metrics associated with samples. In some implementations, each sample comprises a sample of metabolites derived from both the animal subject and the microbiome, and the score or metric associated with the sample comprises a health score of the animal subject.

It will be appreciated that although implementations of the system and method have been described to train a machine learning system, the system and method are not limited to training a machine learning system but may instead be used to identify predictor metabolites without a subsequent training of a machine learning system, which identification may have various advantageous uses. For example, implementations of the systems and methods discussed herein may identify a set of predictor metabolites which are predictive of a state of an animal subject, such as a health, welfare and/or performance state of the animal subject. Such identification may comprise receiving, by a computing device, a plurality of data sets of respective ones of a plurality of animal subjects, wherein each of the plurality of data sets comprises measurement data comprising an indication of a concentration of each of a plurality of metabolites in a sample of a microbiome of a respective animal subject. The measurement data may for example be obtained from an analysis of microbiome samples of the animal subjects, which may for example be sampled from the animal subject's gastrointestinal tract, respiratory tract, oral cavity, skin, or blood. The microbiome samples may for example be intestinal samples, fecal samples, blood samples, skin samplesor saliva samples from the animal subjects. In general, the measurement data may take any suitable form, as for example described elsewhere in this specification with reference to ‘measurements’ or ‘measurement values’. In addition, each animal subject's data set may comprise a label which at least in part characterizes the state of the animal subject. The label may for example characterizes a health state, welfare state and/or performance state of the animal subject. It will be appreciated that a label may not need to provide a complete characterization of an animal subject's health, welfare and/or performance state, but that it may suffice to characterize one or more select aspects of the animal subject's health, welfare and/or performance state. In specific examples, the label may comprise data, such as numerical data, characterizing select aspects of the state such as a growth rate, a body weight gain, a water consumption, a feed consumption, a feed conversion ratio, a lean muscle mass, a weaning weight, a weaning age, an egg production rate, a fertility, a mortality, an infection by a pathogen, a muscular endurance, a methane emission rate, a resting heart rate, a pulmonary arterial pressure, a stress level, a presence or degree of repetitive behavior, a presence or degree of aggressive behavior, hair shedding, feet health of cattle, marbling of meat, of the animal subject.

In a specific example, the label may comprise a score providing a numerical quantification of the state of the animal subject, e.g., of the animal's growth rate, body weight gain, water consumption, etc. Examples of such scores are given elsewhere in this specification. In a specific example of the score, such a score may be a standardized score for a subjective or semi-subjective human characterization of the state of the animal subject. Such a score may thus represent a computer-readable version of the human characterization of the animal subject's state, for example by comprising numeric values which are normalized to a scale.

The implementations of the systems and methods discussed herein may apply a feature selection process to the plurality of data sets to select and thereby identify a subset of the plurality of metabolites of which subset the concentrations are a statistically significant predictor of the state according to the label. An example of such a feature selection process is described elsewhere in this specification, for example with reference to the metabolite analyzer 212, which feature selection process is also known as Boruta. It will be appreciated, however, that the systems and methods discussed herein are not limited to the use of Boruta as feature selection process, but that any other suitable feature selection process may be used instead. In general, such feature selection may, but does not need to, be based on machine learning. It is noted that the concentrations of the subset of the plurality of metabolites may be ‘predictive’ of the state according to the label in that they may be predictive of a value of the label (e.g., growth rate=X), or that the value of the label exceeds or is below a threshold (e.g., growth rate >X), or that the state according to the label is present in an animal subject (e.g., presence of a pathogen), etc.

It will be appreciated that in addition to identifying predictor metabolites, also relations between concentrations of the predictor metabolites and particular values of the animal subject's state may be predicted. For example, once predictor metabolites are identified of which the concentrations are predictive of a growth rate of an animal subjects, e.g., a particular degree thereof, relations may be identified between concentrations of the predictor metabolites and the magnitude of the growth rate. Such relations may in general be linear or non-linear relations, and may in general be identified using known techniques, such as regression analysis. It will be appreciated, however, that such relations do not need to be identified in all applications, for example when the mere presence of predictor metabolites may be sufficiently indicative of the animal subject's state, or when such relations are identified separately from the identification of the predictor metabolites, e.g., by a separate method or system and/or using separate data sets.

By applying the feature selection process to the plurality of data sets of measurement data and labels, a so-called set of predictor metabolites may be identified, which may elsewhere be identified as the ‘subset’ of the plurality of metabolites. Such predictor metabolites have various advantageous uses. For example, having identified a limited number of metabolites of which the concentrations are predictive of, e.g., a positive growth rate, a presence or absence of a pathogen, a reduction in aggressive behavior, etc., microbiome samples may be obtained of other animals, which may be analyzed to obtain measurement data. Such measurement data may in general often be readily obtainable, e.g., from routine checks, or at least obtainable using known techniques. However, labels for the animal state may not routinely be obtained or may represent an additional burden to obtain, e.g., as obtaining labels may require assessment by human experts, e.g., to characterize aggressive behavior, or may require prolonged observation periods, or may require additional types of measurements such as weighting, cardiograph measurements, etc. Nevertheless, using the predictor metabolites, such a label, or in general one or more aspect(s) of the animal subject's state, may be predicted based on the measurement data obtained from the animal subject. As such, while it may be needed to first determine the predictor metabolites of which the concentrations are predictive of a particular animal state, once these metabolites are identified, various applications are within reach in which the state may be predicted from measurement data. Such applications may for example be used in places where extensive assessment or observation of animal subjects is undesirable, for example at a farm. In general, identifying a set of predictor metabolites may take place in a ‘laboratory’ or R&D-type of environment, whilst applications which use the set of predictor metabolites may be used and made available outside of such environments, e.g., at farmers, distributors of feed additives, etc. Here and elsewhere, an ‘application’ may refer to a software application but may also include the general concept of ‘putting the predictor metabolites to use’ in a practical application.

An example of an application which uses a previously identified set of predictor metabolites of which the concentrations are predictive of a particular state of an animal subject may be the prediction of a current or future state of an animal subject based on measurement data of the animal subject. This may involve receiving an identification of the set of predictor metabolites which are predictive of the state of an animal subject and receiving measurement data comprising an identification of concentrations of metabolites in a sample of a microbiome of the animal subject. The measurement data may then be filtered for concentrations of the set of predictor metabolites in the sample. Here, ‘filtering’ may refer to computer-based filtering, i.e., the process of choosing a smaller part of the dataset and using this smaller part for subsequent steps, with the ‘smaller part’ being here the part of the measurement data containing the concentrations of the set of predictor metabolites in the sample, while omitting or disregarding the concentrations of other metabolites in the sample. A current or future state of the animal subject may then be predicted based on the concentrations of the set of predictor metabolites. The current state may for example be predicted based on a current, e.g., most recently obtained, microbiome sample. A future state may for example be predicted based on a difference in the concentrations of the predictor metabolites between at least two measurements over time, e.g., based on longitudinal measurements, which difference may indicate a trend in the state which may be extrapolated to a future time instance to obtain the prediction of the future state.

For example, the current or future state may be predicted by predicting at least one of: a growth rate, a body weight gain, a water consumption, a feed consumption, a feed conversion ratio, a lean muscle mass, a weaning weight, weaning age, an egg production rate, a fertility, a mortality, an infection by a pathogen, a muscular endurance, a methane emission rate, a resting heart rate, a pulmonary arterial pressure, a stress level, a presence or degree of repetitive behavior, a presence or degree of aggressive behavior, a presence or degree of hair shedding, a characteristic of feet of cattle, a presence or degree of marbling of meat, of the animal subject.

Another example of an application which uses a previously identified set of predictor metabolites of which the concentrations are predictive of a particular state of an animal subject may be the monitoring a state of an animal subject. This may involve receiving an identification of the set of predictor metabolites which are predictive of the state of an animal subject and receiving measurement data comprising an identification of concentrations of metabolites in a sample of a microbiome of the animal subject. The measurement data may then be filtered for concentrations of the set of predictor metabolites in the sample. The state of the animal subject may then be monitored by comparing the one or more concentrations against one or more reference concentration for the respective predictor metabolites. In some embodiments, an output signal may be generated, such as a warning signal or a control signal, which may be indicative of one or more of the concentrations of respective predictor metabolites in the microbiome sample corresponding to or deviating from the one or more reference concentration. This way, action may be taken based on the output signal, e.g., manually by human intervention or automatically, for example to take measures so that the state of the animal subject is positively affected.

Another example of an application is similar to the abovementioned monitoring but may use a machine learning system which is trained on training data which comprises the measurement data corresponding to the predictor metabolites (elsewhere also referred to as ‘filtered’ data) and the labels. The training of such a machine learning system is described elsewhere in this specification. In such an application, a machine learning system may be obtained which is trained in a manner as described in this specification. Furthermore, measurement data comprising an identification of concentrations of metabolites in a sample of a microbiome of the animal subject may be obtained. The machine learning system may then be ‘applied’ to the measurement data to predict a state of the animal subject. Having predicted the state, an output signal may be generated, such as a warning signal or a control signal, which may be indicative of the predicted state deviating, or conversely not deviating, from a reference state.

In some embodiments, when identifying the predictor metabolites, the measurement data may be obtained from microbiome samples of animal subjects, and in particular from two types of animal subjects: animal subjects belonging to a test group and animal subjects belonging to a control group. This may relate to the following: a test group of animal subjects may be subjected to a stimulus to affect a state of the animal subjects, or a test group of animal subjects may be provided which is already subjected to the stimulus, e.g., without actively applying the stimulus. In addition, a control group of animal subjects may be provided which are not subjected to the stimulus. The measurement data may be obtained from both groups of animal subjects. The label, which may be used for identifying the predictor metabolites, may be indicative of whether a respective animal subject is part of the test group or the control group of animal subjects. As such, predictor metabolites may be identified of which the concentrations are predictive of whether an animal subject has been, or is being subjected to the stimulus.

Such a stimulus may be applied purposefully to the test group of animal subjects, for example by supplying a nutritional additive to feed and/or drinking water of the test group of animal subjects, controlling an environmental parameter of an environment of the test group of animal subjects, controlling a size and/or type of space in which the test group of animal subjects are kept, controlling a density of animal subjects in the test group of animal subjects; and controlling access of the test group of animal subjects to an outside environment. The control group of animal subjects may differ from the test group in that no, or a different nutritional additive may be provided, the environmental parameter may be controlled differently or not at all, the size and/or type of space in which the control group of animals is kept may be different from that of the test group, the density of animal subjects in the control group may be different from that in the test group, the access of the control group of animal subjects to the outside environment may be different from that of the test group, etc. In general, the stimulus may be represented by a difference in how the test group and the control group of animals are treated, kept, etc. With continued reference to the environmental parameter: such a parameter may for example comprise a parameter controlling an aspect of a light regime (such as a light level, a light duration, a light spectrum, etc.) to which the animal subjects are subjected, a temperature in the animal subject's environment, air pollution in the animal subject's environment and a humidity in the animal subject's environment. It will be appreciated that a stimulus may also be a stimulus which is not purposefully applied. For example, the test group of animals may be a group of animals which are or have been subjected to a pathogen, for example by there being an uncontrolled infection of the animal subjects. In general, the state of the animal subject, which may be predicted, may be ‘stimulus applied’ or ‘stimulus not applied’. The prediction of such a state may have various advantages uses.

In some examples, the control group and the test group of animal subjects may contain substantially the same animal subjects, but with the control group being a group of animal subjects before application of the stimulus and the test group being the group of animal subjects after application of the stimulus. The measurement data may thus be obtained by obtaining microbiome samples, e.g., from fecal matter, before and after application of the stimulus.

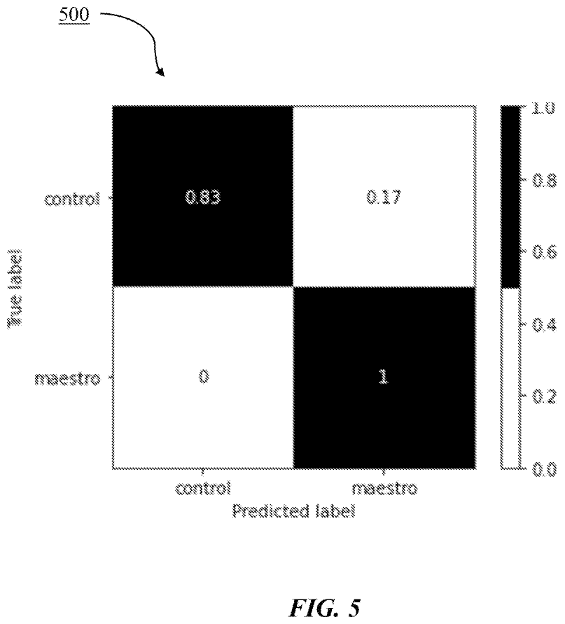

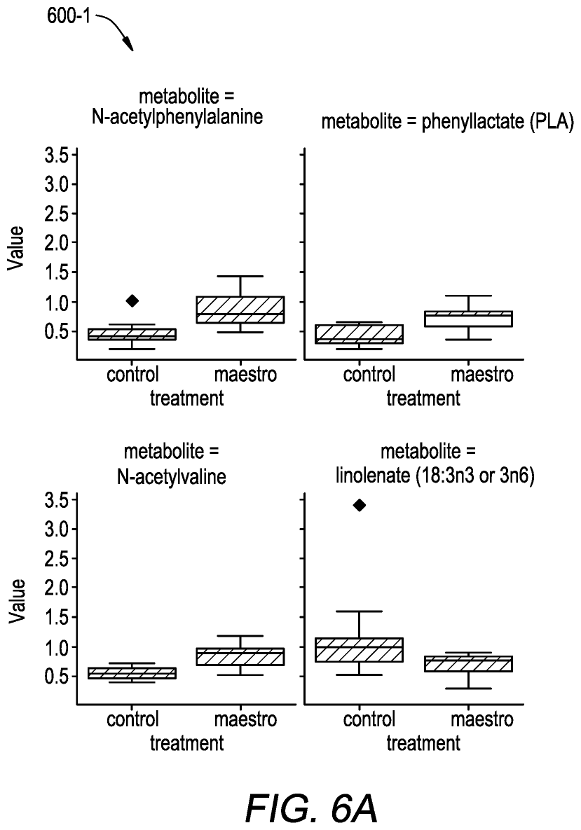

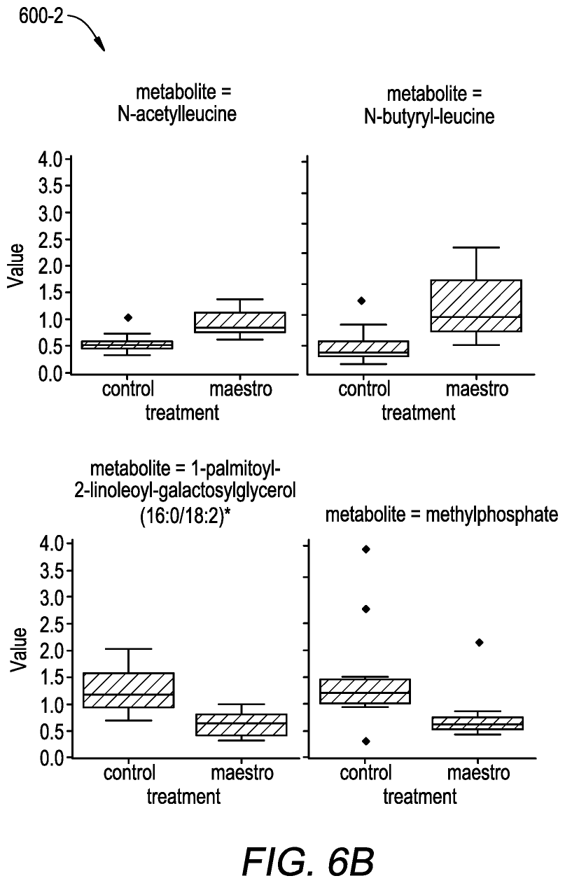

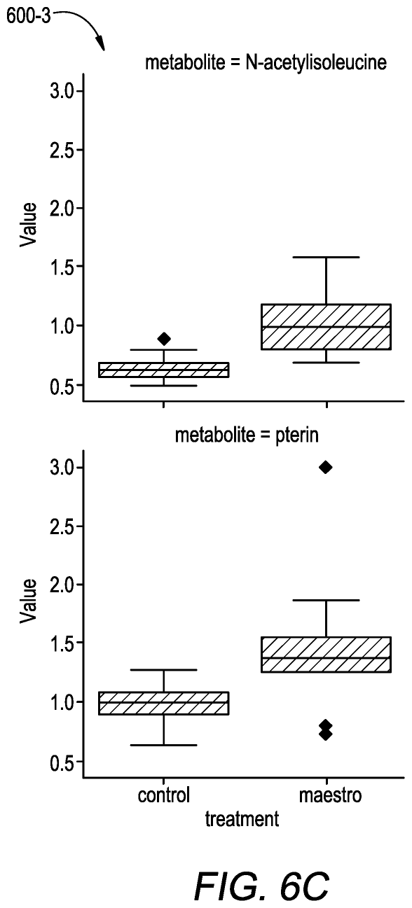

FIG. 5 relates to an example where a set of predictor metabolites are identified of which the concentrations are predictive of an animal subject having been subjected to a stimulus. In this example, the stimulus is the supplementation of nutritional additives in the form of maestro, being a microbiome metabolic modulator in form of a complex which contains multiple oligosaccharide compounds and which is further described in WO2020097454 (hereby incorporated by reference). Maestro is able to increase Phenyllactate (PLA) which is known for its benefit in animal metabolisms. In this example, Maestro was fed as a nutritional supplement to a group of 18 chickens, while a control group of 20 chickens was not fed maestro. Fecal samples were obtained from both groups. A label was generated indicating whether the measurement data of a fecal sample belonged to a chicken from the control group or from the test group (also referred to as ‘maestro group’). FIG. 5 shows the result of the identification of a set of predictor metabolites of which their concentrations are indicative of a treatment by maestro in form of a confusion matrix 500. Here, a Python implementation of Boruta with 94 iterations was used. It can be seen from the confusion matrix 500 the true positive rate (a sample from the maestro group is identified as such) is 1 (100%) while the true negative rate (a sample for the control group is identified as such) is 0.83 (83%). Moreover, the false positive rate (a sample from the control group is identified as belonging to the maestro group) is 0.17 (17%) and the false negative rate (a sample from the maestro group is identified as belonging to the control group) is 0. FIGS. 6A-6C show the found predictor metabolites in the form of a number of graphs 600-1, 600-2, 600-3, showing for each of the predictor metabolites the concentrations which are found to be predictive of treatment by maestro, including their confidence intervals. In total, 10 predictor metabolites were identified from 845 metabolites in the sample. The overall accuracy of being able to predict whether a chicken was treated with maestro has been found to be 86%.

An example of an application which uses a previously identified set of predictor metabolites of which the concentrations are predictive of an animal subject having been subjected to a stimulus may be the following in which a metabolic mechanism or mode of action of a stimulus affecting the state of an animal subject identified. This may involve receiving an identification of a set of predictor metabolites which are predictive of the state of the animal subject, wherein the set of predictor metabolites are identified using a test group of animal subjects subjected to a stimulus and a control group of animal subjects not subjected to the stimulus. Using the set of predictor metabolites, one or more metabolic pathways associated with the set of the plurality of metabolites may be identified. Such identification may make use of known relations between metabolites and their pathways, e.g., as previously identified in scientific literature, or may be newly identified, e.g., based on research and analysis effort. Having identified the one or more pathways, a metabolic mechanism or mode of action of the stimulus may be identified. Such identified metabolic mechanism or mode of action of the stimulus may in turn also have various advantageous uses. For example, based on said identified metabolic mechanism or mode of action of the stimulus, a type and/or a concentration of one or more nutritional additives may be determined which, when ingested by the animal subject, generate the effect of the stimulus on the state of the animal subject. Effectively, the nutritional additive(s) may be determined to mimic the effect of the stimulus on the state of the animal subject. This may be advantageous in case the application of the stimulus has other drawbacks, for example in terms of complexity, cost, or if it is not possible to apply the stimulus in certain situations. Here, the nutritional additive(s) may be used instead of the stimulus. Such nutritional additives may for example be fed to the animal subjects in form of a nutritional supplement of which the composition may be determined to comprise the one or more nutritional additives. In some examples, where the application of the stimulus represents a treatment, e.g., by subjecting animal subjects to a particular light regime, animal subjects may be treated, namely by mimicking the stimulus by supplying the one or more nutritional additives, or a nutritional supplement comprising said additive(s), to feed and/or drinking water of animal subjects.

Another example of an application which uses a previously identified set of predictor metabolites of which the concentrations are predictive of an animal subject having been subjected to a stimulus may be the following, in which a presence of a pathogen affecting a state of an animal subject is identified. This may involve receiving an identification of a set of predictor metabolites which are predictive of the state of the animal subject, wherein the set of predictor metabolites are identified using a test group of animal subjects subjected to a pathogen and a control group of animal subjects not subjected to the pathogen. Furthermore, measurement data may be received comprising an identification of concentrations of metabolites in a sample of a microbiome of the animal subject. The measurement data may then be filtered for concentrations of the set of predictor metabolites in the sample, and the presence of the pathogen in the animal subject may be predicted based on these predictor metabolite concentrations.

It will be appreciated that while the metabolite analysis and prediction as described in this specification may be computer-implemented, other steps, such as subjecting a test group of animal subjects to a stimulus, providing a control group of animal subjects, obtaining and analyzing microbiome samples, etc., may not be or may not need to be computer-implemented.

It will be appreciated that while the metabolite analysis and prediction as described in this specification may be described to be applied to animal subjects, such metabolite analysis and prediction may also be applied to mammal subjects other than animals, that is, to human subjects. Accordingly, any embodiment described in this specification, including embodiments defined by the claims or clauses, which are applied to animal subjects, may equally be applied to human subjects, or in general to mammal subject, unless otherwise noted or precluded.

### B. Computing Environment

Having discussed specific embodiments of the present solution, it may be helpful to describe aspects of the operating environment as well as associated system components (e.g., hardware elements) in connection with the methods and systems described herein.

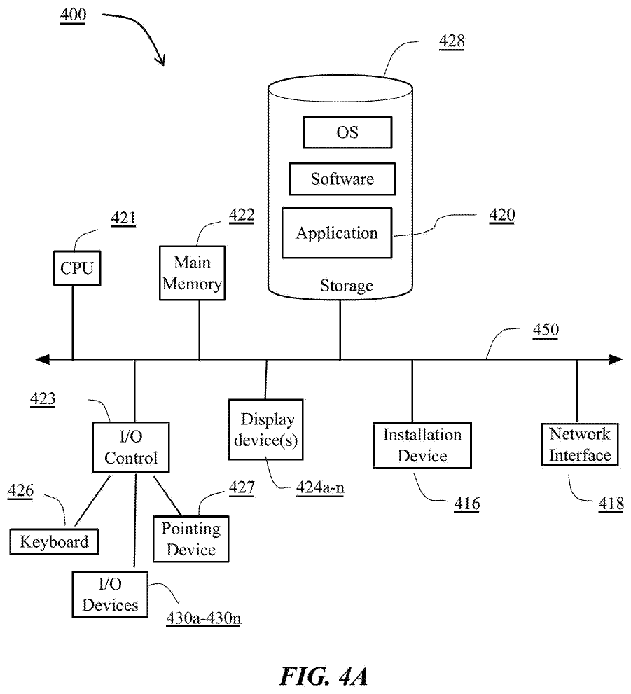

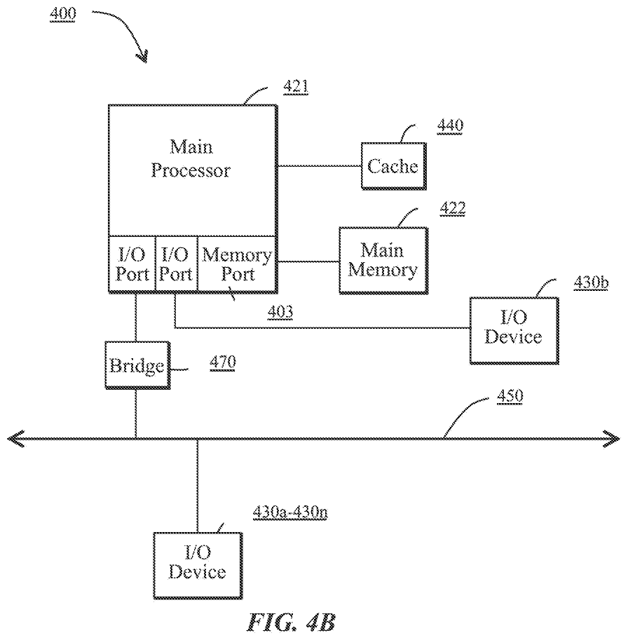

The systems discussed herein may be deployed as and/or executed on any type and form of computing device, such as a computer, network device or appliance capable of communicating on any type and form of network and performing the operations described herein. FIGS. 4A and 4B depict block diagrams of a computing device 400 useful for practicing an embodiment of the wireless communication devices 402 or the access point 406. As shown in FIGS. 4A and 4B, each computing device 400 includes a central processing unit 421, and a main memory unit 422. As shown in FIG. 4A, a computing device 400 may include a storage device 428, an installation device 416, a network interface 418, an I/O controller 423, display devices 424a-424n, a keyboard 426 and a pointing device 427, such as a mouse. The storage device 428 may include, without limitation, an operating system and/or software. As shown in FIG. 4B, each computing device 400 may also include additional optional elements, such as a memory port 403, a bridge 470, one or more input/output devices 430a-430n (generally referred to using reference numeral 430), and a cache memory 440 in communication with the central processing unit 421.

The central processing unit 421 is any logic circuitry that responds to and processes instructions fetched from the main memory unit 422. In many embodiments, the central processing unit 421 is provided by a microprocessor unit, such as: those manufactured by Intel Corporation of Mountain View, California; those manufactured by International Business Machines of White Plains, New York; or those manufactured by Advanced Micro Devices of Sunnyvale, California. The computing device 400 may be based on any of these processors, or any other processor capable of operating as described herein.

Main memory unit 422 may be one or more memory chips capable of storing data and allowing any storage location to be directly accessed by the microprocessor 421, such as any type or variant of Static random access memory (SRAM), Dynamic random access memory (DRAM), Ferroelectric RAM (FRAM), NAND Flash, NOR Flash and Solid State Drives (SSD). The main memory 422 may be based on any of the above described memory chips, or any other available memory chips capable of operating as described herein. In the embodiment shown in FIG. 4A, the processor 421 communicates with main memory 422 via a system bus 450 (described in more detail below). FIG. 4B depicts an embodiment of a computing device 400 in which the processor communicates directly with main memory 422 via a memory port 403. For example, in FIG. 4B the main memory 422 may be DRDRAM.

FIG. 4B depicts an embodiment in which the main processor 421 communicates directly with cache memory 440 via a secondary bus, sometimes referred to as a backside bus. In other embodiments, the main processor 421 communicates with cache memory 440 using the system bus 450. Cache memory 440 typically has a faster response time than main memory 422 and is provided by, for example, SRAM, BSRAM, or EDRAM. In the embodiment shown in FIG. 4B, the processor 421 communicates with various I/O devices 430 via a local system bus 450. Various buses may be used to connect the central processing unit 421 to any of the I/O devices 430, for example, a VESA VL bus, an ISA bus, an EISA bus, a MicroChannel Architecture (MCA) bus, a PCI bus, a PCI-X bus, a PCI-Express bus, or a NuBus. For embodiments in which the I/O device is a video display 424, the processor 421 may use an Advanced Graphics Port (AGP) to communicate with the display 424. FIG. 4B depicts an embodiment of a computer 400 in which the main processor 421 may communicate directly with I/O device 430b, for example via HYPERTRANSPORT, RAPIDIO, or INFINIBAND communications technology. FIG. 4B also depicts an embodiment in which local busses and direct communication are mixed: the processor 421 communicates with I/O device 430a using a local interconnect bus while communicating with I/O device 430b directly.

A wide variety of I/O devices 430a-430n may be present in the computing device 400. Input devices include keyboards, mice, trackpads, trackballs, microphones, dials, touch pads, touch screen, and drawing tablets. Output devices include video displays, speakers, inkjet printers, laser printers, projectors and dye-sublimation printers. The I/O devices may be controlled by an I/O controller 423 as shown in FIG. 4A. The I/O controller may control one or more I/O devices such as a keyboard 426 and a pointing device 427, e.g., a mouse or optical pen. Furthermore, an I/O device may also provide storage and/or an installation medium 416 for the computing device 400. In still other embodiments, the computing device 400 may provide USB connections (not shown) to receive handheld USB storage devices such as the USB Flash Drive line of devices manufactured by Twintech Industry, Inc. of Los Alamitos, California.

Referring again to FIG. 4A, the computing device 400 may support any suitable installation device 416, such as a disk drive, a CD-ROM drive, a CD-R/RW drive, a DVD-ROM drive, a flash memory drive, tape drives of various formats, USB device, hard-drive, a network interface, or any other device suitable for installing software and programs. The computing device 400 may further include a storage device, such as one or more hard disk drives or redundant arrays of independent disks, for storing an operating system and other related software, and for storing application software programs such as any program or software 420 for implementing (e.g., configured and/or designed for) the systems and methods described herein. Optionally, any of the installation devices 416 could also be used as the storage device. Additionally, the operating system and the software can be run from a bootable medium.

Furthermore, the computing device 400 may include a network interface 418 to interface to the network 404 through a variety of connections including, but not limited to, standard telephone lines, LAN or WAN links (e.g., 802.11, T1, T3, 56 kb, X.25, SNA, DECNET), broadband connections (e.g., ISDN, Frame Relay, ATM, Gigabit Ethernet, Ethernet-over-SONET), wireless connections, or some combination of any or all of the above. Connections can be established using a variety of communication protocols (e.g., TCP/IP, IPX, SPX, NetBIOS, Ethernet, ARCNET, SONET, SDH, Fiber Distributed Data Interface (FDDI), RS232, IEEE 802.11, IEEE 802.11a, IEEE 802.11b, IEEE 802.11g, IEEE 802.11n, IEEE 802.11ac, IEEE 802.11ad, CDMA, GSM, WiMax and direct asynchronous connections). In one embodiment, the computing device 400 communicates with other computing devices 400′ via any type and/or form of gateway or tunneling protocol such as Secure Socket Layer (SSL) or Transport Layer Security (TLS). The network interface 418 may include a built-in network adapter, network interface card, PCMCIA network card, card bus network adapter, wireless network adapter, USB network adapter, modem or any other device suitable for interfacing the computing device 400 to any type of network capable of communication and performing the operations described herein.

In some embodiments, the computing device 400 may include or be connected to one or more display devices 424a-424n. As such, any of the I/O devices 430a-430n and/or the I/O controller 423 may include any type and/or form of suitable hardware, software, or combination of hardware and software to support, enable or provide for the connection and use of the display device(s) 424a-424n by the computing device 400. For example, the computing device 400 may include any type and/or form of video adapter, video card, driver, and/or library to interface, communicate, connect or otherwise use the display device(s) 424a-424n. In one embodiment, a video adapter may include multiple connectors to interface to the display device(s) 424a-424n. In other embodiments, the computing device 400 may include multiple video adapters, with each video adapter connected to the display device(s) 424a-424n. In some embodiments, any portion of the operating system of the computing device 400 may be configured for using multiple displays 424a-424n. One ordinarily skilled in the art will recognize and appreciate the various ways and embodiments that a computing device 400 may be configured to have one or more display devices 424a-424n.

In further embodiments, an I/O device 430 may be a bridge between the system bus 450 and an external communication bus, such as a USB bus, an Apple Desktop Bus, an RS-232 serial connection, a SCSI bus, a FireWire bus, a FireWire 800 bus, an Ethernet bus, an AppleTalk bus, a Gigabit Ethernet bus, an Asynchronous Transfer Mode bus, a FibreChannel bus, a Serial Attached small computer system interface bus, a USB connection, or a HDMI bus.

A computing device 400 of the sort depicted in FIGS. 4A and 4B may operate under the control of an operating system, which control scheduling of tasks and access to system resources. The computing device 400 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: Android, produced by Google Inc.; WINDOWS 7, 8, 10, 11 produced by Microsoft Corporation of Redmond, Washington; MAC OS, produced by Apple Computer of Cupertino, California; WebOS, produced by Research In Motion (RIM); OS/2, produced by International Business Machines of Armonk, New York; and Linux, a freely-available operating system distributed by Caldera Corp. of Salt Lake City, Utah, or any type and/or form of a Unix operating system, among others.

The computer system 400 can be any workstation, telephone, desktop computer, laptop or notebook computer, server, handheld computer, mobile telephone or other portable telecommunications device, media playing device, a gaming system, mobile computing device, or any other type and/or form of computing, telecommunications or media device that is capable of communication. The computer system 400 has sufficient processor power and memory capacity to perform the operations described herein.

In some embodiments, the computing device 400 may have different processors, operating systems, and input devices consistent with the device. For example, in one embodiment, the computing device 400 is a smart phone, mobile device, tablet or personal digital assistant. In still other embodiments, the computing device 400 is an Android-based mobile device, an iPhone smart phone manufactured by Apple Computer of Cupertino, California, or a Blackberry or WebOS-based handheld device or smart phone, such as the devices manufactured by Research In Motion Limited. Moreover, the computing device 400 can be any workstation, desktop computer, laptop or notebook computer, server, handheld computer, mobile telephone, any other computer, or other form of computing or telecommunications device that is capable of communication and that has sufficient processor power and memory capacity to perform the operations described herein.

Although the disclosure may reference one or more “users”, such “users” may refer to user-associated devices or stations (STAs), for example, consistent with the terms “user” and “multi-user” typically used in the context of a multi-user multiple-input and multiple-output (MU-MIMO) environment.

Although examples of communications systems described above may include devices and APs operating according to an 802.11 standard, it should be understood that embodiments of the systems and methods described can operate according to other standards and use wireless communications devices other than devices configured as devices and APs. For example, multiple-unit communication interfaces associated with cellular networks, satellite communications, vehicle communication networks, and other non-802.11 wireless networks can utilize the systems and methods described herein to achieve improved overall capacity and/or link quality without departing from the scope of the systems and methods described herein.

It should be noted that certain passages of this disclosure may reference terms such as “first” and “second” in connection with devices, mode of operation, transmit chains, antennas, etc., for purposes of identifying or differentiating one from another or from others. These terms are not intended to merely relate entities (e.g., a first device and a second device) temporally or according to a sequence, although in some cases, these entities may include such a relationship. Nor do these terms limit the number of possible entities (e.g., devices) that may operate within a system or environment.

It should be understood that the systems described above may provide multiple ones of any or each of those components and these components may be provided on either a standalone machine or, in some embodiments, on multiple machines in a distributed system. In addition, the systems and methods described above may be provided as one or more computer-readable programs or executable instructions embodied on or in one or more articles of manufacture. The article of manufacture may be a floppy disk, a hard disk, a CD-ROM, a flash memory card, a PROM, a RAM, a ROM, or a magnetic tape. In general, the computer-readable programs may be implemented in any programming language, such as LISP, PERL, C, C++, C #, PROLOG, or in any byte code language such as JAVA. The software programs or executable instructions may be stored on or in one or more articles of manufacture as object code.

While the foregoing written description of the methods and systems enables one of ordinary skill to make and use what is considered presently to be the best mode thereof, those of ordinary skill will understand and appreciate the existence of variations, combinations, and equivalents of the specific embodiment, method, and examples herein. The present methods and systems should therefore not be limited by the above described embodiments, methods, and examples, but by all embodiments and methods within the scope and spirit of the disclosure.

The following clauses define further implementations of the systems and methods discussed herein which may be separately claimed.

Clause 1. A method for pre-processing metabolite data for machine learning-based analysis, comprising:
receiving, by a computing device, a plurality of initial data sets, each data set comprising an identification of a concentration of each of a plurality of metabolites in a sample and a score associated with the sample;
creating, by the computing device, a corresponding plurality of additional data sets, each additional data set comprising the score from a corresponding initial data set and a random resorting of the concentrations of each of the plurality of metabolites from the corresponding initial data set;
generating, by the computing device via a first classifier using the plurality of additional data sets, a first importance score for each of the plurality of metabolites;
identifying, by the computing device, a maximum first importance score of the plurality of metabolites generated using the plurality of additional data sets;
generating, by the computing device via the first classifier using the plurality of initial data sets, a second importance score for each of the plurality of metabolites;
selecting, by the computing device, a subset of the plurality of metabolites with second importance scores exceeding the maximum first importance score;
filtering, by the computing device, the identifications of the concentrations of each of the plurality of metabolites of the plurality of initial data sets according to the selected subset of the plurality of metabolites; and
training, by the computing device using the filtered plurality of initial data sets, a machine learning system to predict scores associated with samples.
   Clause 2. The method of claim 1, wherein each sample comprises a sample of metabolites derived from both the subject and the microbiome, and wherein the score associated with the sample comprises a health score of the subject.
   Clause 3. The method of clause 2, wherein the microbiome of the subject is sampled from the subject's gastrointestinal tract and wherein the health score is a fecal score or animal performance score.
   Clause 4. The method of clause 2, wherein the microbiome of the subject is sampled from the subject's blood and wherein the health score is an animal performance score.

Clause 5. The method of clause 2, further comprising:
predicting, by the computing device using the trained machine learning system, a health score above a threshold for a new sample; and
providing a control signal, by the computing device to an automated feeding system responsive to the predicted health score being above the threshold, to modify a supplement concentration for the subject.
   Clause 6. The method of clause 1, wherein filtering the identifications of the concentrations of each of the plurality of metabolites of the plurality of initial data sets further comprises removing, from the initial data set, identifications of metabolites associated with second importance scores that are equal to or less than the maximum first importance score.
   Clause 7. The method of clause 1, wherein the machine learning system comprises a neural network; and wherein training the neural network further comprises providing the filtered plurality of initial data sets to the neural network in a supervised learning process.
   Clause 8. The method of clause 1, further comprising identifying, within a metabolic network comprising nodes corresponding to metabolites and edges corresponding to enzymes converting between metabolites, one or more metabolites connected via an edge to at least one metabolite of the selected subset of the plurality of metabolites with second importance scores exceeding the maximum first importance score.
   Clause 9. The method of clause 8, further comprising recording, to a data structure stored in a memory of the computing device, the identified one or more metabolites.
   Clause 10. A system for pre-processing metabolite data for machine learning-based analysis, comprising:
a computing device comprising a processor executing a first classifier and a machine learning engine;
wherein the processor is configured to:
receive a plurality of initial data sets, each data set comprising an identification of a concentration of each of a plurality of metabolites in a sample and a score associated with the sample,
create a corresponding plurality of additional data sets, each additional data set comprising the score from a corresponding initial data set and a random resorting of the concentrations of each of the plurality of metabolites from the corresponding initial data set,
generate, via the first classifier using the plurality of additional data sets, a first importance score for each of the plurality of metabolites,
identify a maximum first importance score of the plurality of metabolites generated using the plurality of additional data sets,
generate, via the first classifier using the plurality of initial data sets, a second importance score for each of the plurality of metabolites,
select a subset of the plurality of metabolites with second importance scores exceeding the maximum first importance score,
filter the identifications of the concentrations of each of the plurality of metabolites of the plurality of initial data sets according to the selected subset of the plurality of metabolites, and
train, using the filtered plurality of initial data sets, the machine learning system to predict scores associated with samples.
   Clause 11. The system of clause 10, wherein each sample comprises a sample of metabolites derived from both the subject and the microbiome, and wherein the score associated with the sample comprises a health score of the subject.
   Clause 12. The system of clause 11, wherein the microbiome of the subject is a fecal sample and wherein the health score is a fecal score.
   Clause 13. The method of clause 11, wherein the microbiome of the subject is sampled from the subject's blood and wherein the health score is an animal performance score.
   Clause 14. The system of clause 12 or 13, wherein the processor is further configured to:
predict, using the trained machine learning system, a fecal score above a threshold for a new fecal sample; and
provide a control signal, to an automated feeding system responsive to the predicted fecal score being above the threshold, to modify a supplement concentration for the subject.
   Clause 15. The system of clause 10, wherein the processor is further configured to remove, from the initial data set, identifications of metabolites associated with second importance scores that are equal to or less than the maximum first importance score.
   Clause 16. The system of clause 10, wherein the machine learning system comprises a neural network.
   Clause 17. The system of clause 16, wherein the processor is further configured to provide the filtered plurality of initial data sets to the neural network in a supervised learning process.
   Clause 18. The system of clause 10, wherein the processor is further configured to identify, within a metabolic network comprising nodes corresponding to metabolites and edges corresponding to enzymes converting between metabolites, one or more metabolites connected via an edge to at least one metabolite of the selected subset of the plurality of metabolites with second importance scores exceeding the maximum first importance score.
   Clause 19. The system of clause 18, wherein the computing device further comprises a memory, and wherein the processor is further configured to record, to a data structure stored in the memory, the identified one or more metabolites.
   Clause 20. A non-transitory computer readable medium comprising one or more instructions, the execution of which cause a processor of a computing device to:
receive a plurality of initial data sets, each data set comprising an identification of a concentration of each of a plurality of metabolites in a sample and a score associated with the sample,
create a corresponding plurality of additional data sets, each additional data set comprising the score from a corresponding initial data set and a random resorting of the concentrations of each of the plurality of metabolites from the corresponding initial data set,
generate, via a first classifier using the plurality of additional data sets, a first importance score for each of the plurality of metabolites,
identify a maximum first importance score of the plurality of metabolites generated using the plurality of additional data sets,
generate, via the first classifier using the plurality of initial data sets, a second importance score for each of the plurality of metabolites,
select a subset of the plurality of metabolites with second importance scores exceeding the maximum first importance score,
filter the identifications of the concentrations of each of the plurality of metabolites of the plurality of initial data sets according to the selected subset of the plurality of metabolites, and train, using the filtered plurality of initial data sets, a machine learning system to predict scores associated with samples.
   Clause 21. The computer readable medium of clause 20, wherein each sample comprises a sample of metabolites derived from both the subject and the microbiome, and wherein the score associated with the sample comprises a health score of the subject.

1-39. (canceled)

40. A method of identifying a set of predictor metabolites which are predictive of a state of a subject being an animal subject, comprising:
receiving, by a computing device, a plurality of data sets of respective ones of a plurality of subjects, wherein each of the plurality of data sets comprises:
measurement data comprising an indication of a concentration of each of a plurality of metabolites in a sample of a microbiome of a respective subject, and
a label at least in part characterizing the state of the subject;
applying, by the computing device, a feature selection process to the plurality of data sets to select and thereby identify a subset of the plurality of metabolites of which subset the concentrations are a statistically significant predictor of the state according to the label.

41. The method of claim 40, wherein the measurement data is obtained by:
subjecting a test group of animal subjects to a stimulus to affect a state of the animal subjects, or providing a test group of animal subjects subjected to the stimulus, and
providing a control group of animal subjects which are not subjected to the stimulus; and
wherein the label is indicative of whether a respective animal subject is part of the test group or the control group of animal subjects.

42. The method of claim 41, wherein subjecting the test group of animal subjects to the stimulus comprises at least one of:
supplying a nutritional additive to feed and/or drinking water of the test group of animal subjects;
topically administering a composition comprising a skin-care active to the skin of the test group of animal subjects;
subjecting the test group of animal subjects to a pathogen;
controlling an environmental parameter of an environment of the test group of animal subjects;
controlling a size and/or type of space in which the test group of animal subjects are kept;
controlling a density of animal subjects in the test group of animal subjects; and
controlling access of the test group of animal subjects to an outside environment.

43. The method of claim 40, wherein the label characterizes a health state, welfare state or performance state of the subject, a growth rate, a body weight gain, a water consumption, a feed consumption, a feed conversion ratio, a lean muscle mass, a weaning weight, a weaning age, an egg production rate, a fertility, a mortality, an infection by a pathogen, a muscular endurance, a methane emission rate, a resting heart rate, a pulmonary arterial pressure, a stress level, a presence or degree of repetitive behavior, a presence or degree of aggressive behavior, hair shedding, feet health of cattle, marbling of meat, skin age, skin moisturization, skin sebum, skin barrier (TEWL), skin elasticity, skin oiliness, skin appearance and/or skin glow, of the subject.

44. A method of determining whether an animal subject has been or is being subjected to a stimulus, comprising identifying a set of predictor metabolites by the method of claim 40, wherein the set of predictor metabolites comprises at least one, preferably at least two, three, four, five, six, seven, eight, nine, or even ten predictor metabolite(s) selected from N-acetylphenylalanine; phenyllactate (PLA); N-acetylvaline; linolenate (18:3n3 or 3n6); N-acetylleucine; N-butyryl-leucine; N-acetylisoleucine; pterin; 1-palmitoyl-2-linoleoyl-galactosylglycerol (16:0/18:2); and methylphosphate.

45. A method of identifying a metabolic mechanism or mode of action of a stimulus affecting a state of an animal subject, the method comprising:
receiving an identification of a set of predictor metabolites which are predictive of the state of the animal subject, wherein the set of predictor metabolites are identified by the method of claim 41 using a test group of animal subjects subjected to a stimulus;
identifying one or more metabolic pathways associated with the set of the plurality of metabolites;
based on said identified one or more pathways, identifying a metabolic mechanism or mode of action of the stimulus.

46. The method of claim 45, further comprising, based on said identified metabolic mechanism or mode of action of the stimulus, determining a type and/or a concentration of one or more nutritional additives which, when ingested by the animal subject, generate the effect of the stimulus on the state of the animal subject, preferably wherein the set of predictor metabolites comprises N-acetylphenylalanine; phenyllactate (PLA); N-acetylvaline; linolenate (18:3n3 or 3n6); N-acetylleucine; N-butyryl-leucine; N-acetylisoleucine; pterin; 1-palmitoyl-2-linoleoyl-galactosylglycerol (16:0/18:2); and/or methylphosphate.

47. A method of determining whether an animal subject has been or is being subjected to a stimulus, comprising
identifying a set of predictor metabolites by the method of claim 40,
wherein the set of predictor metabolites comprises at least one, preferably at least two, three, four, five, six, seven, eight, nine, or even ten predictor metabolite(s) selected from N-acetylphenylalanine; phenyllactate (PLA); N-acetylvaline; linolenate (18:3n3 or 3n6); N-acetylleucine; N-butyryl-leucine; N-acetylisoleucine; pterin; 1-palmitoyl-2-linoleoyl-galactosylglycerol (16:0/18:2); and methylphosphate.

48. A method of treating an animal subject by supplying a nutritional additive as determined by the method of claim 46 to feed and/or drinking water of an animal subject.

49. A method of predicting a current or future state of an animal subject, comprising:
receiving, by a computing device, an identification of a set of predictor metabolites which are predictive of a state of an animal subject, wherein the set of predictor metabolites are identified by the method of claim 40;
receiving, by the computing device, measurement data comprising an identification of concentrations of metabolites in a sample of a microbiome of the animal subject;
filtering, by the computing device, the measurement data for concentrations of the set of predictor metabolites in the sample; and
predicting, by the computing device, the current or future state of the animal subject based on the concentrations of the set of predictor metabolites.

50. A method of identifying a presence of a pathogen affecting a state of an animal subject, the method comprising:
receiving, by a computing device, an identification of a set of predictor metabolites which are predictive of the state of the animal subject, wherein the set of predictor metabolites are identified by the method of claim 41 using a test group of animal subjects subjected to a pathogen;
receiving, by the computing device, measurement data comprising an identification of concentrations of metabolites in a sample of a microbiome of the animal subject;
filtering, by the computing device, the measurement data for concentrations of the set of predictor metabolites in the sample; and
predicting, by the computing device, a presence of the pathogen in the animal subject based on the concentrations of the set of predictor metabolites.

51. A method of monitoring a state of an animal subject, comprising:
receiving, by a computing device, an identification of a set of predictor metabolites which are predictive of a state of an animal subject, wherein the set of predictor metabolites are identified by the method of claim 40;
receiving, by the computing device, measurement data comprising an identification of concentrations of metabolites in a sample of a microbiome of the animal subject;
filtering, by the computing device, the measurement data for concentrations of the set of predictor metabolites in the sample; and
providing, by the computing device, an output signal which is indicative of one or more of the concentrations of respective predictor metabolites corresponding to or deviating from one or more reference concentration for the respective predictor metabolites.

52. A non-transitory computer readable medium comprising one or more instructions, the execution of which cause a processor of a computing device to perform the method of claim 40.

53. A system for identifying a set of predictor metabolites which are predictive of a state of a subject being a mammal subject, comprising:
a computing device comprising a processor configured to:
receive a plurality of data sets of respective ones of a plurality of subjects, wherein each of the plurality of data sets comprises:
measurement data comprising an indication of a concentration of each of a plurality of metabolites in a sample of a microbiome of a respective subject, and
a label at least in part characterizing the state of the subject;
apply a feature selection process to the plurality of data sets to select and thereby identify a subset of the plurality of metabolites of which subset the concentrations are a statistically significant predictor of the state according to the label.

54. A system for identifying a metabolic mechanism or mode of action of a stimulus affecting a state of an animal subject, comprising:
a computing device comprising a processor configured to:
receive an identification of a set of predictor metabolites which are predictive of the state of the animal subject, wherein the set of predictor metabolites are identified by the method of claim 41 using a test group of animal subjects subjected to a stimulus;
identify one or more metabolic pathways associated with the subset of the plurality of metabolites;
based on said identified one or more pathways, identify a metabolic mechanism or mode of action of the stimulus.

55. A system for predicting a current or future state of an animal subject, comprising:
a computing device comprising a processor configured to:
receive an identification of a set of predictor metabolites which are predictive of a state of an animal subject, wherein the set of predictor metabolites are identified by the method of claim 40;
receive measurement data comprising an identification of concentrations of metabolites in a sample of a microbiome of the animal subject;
filter the measurement data for concentrations of the set of predictor metabolites in the sample; and
predict the current or future state of the animal subject based on the concentrations of the set of predictor metabolites.

56. A system for identifying a presence of a pathogen affecting a state of an animal subject, comprising:
a computing device comprising a processor configured to:
receive an identification of a set of predictor metabolites which are predictive of the state of the animal subject, wherein the set of predictor metabolites are identified by the method of claim 41 using a test group of animal subjects subjected to a pathogen;
receive measurement data comprising an identification of concentrations of metabolites in a sample of a microbiome of the animal subject;
filter the measurement data for concentrations of the set of predictor metabolites in the sample; and
predict a presence of the pathogen in the animal subject based on the concentrations of the set of predictor metabolites.

57. A system for monitoring a state of an animal subject, comprising:
a computing device comprising a processor configured to:
receive an identification of a set of predictor metabolites which are predictive of a state of an animal subject, wherein the set of predictor metabolites are identified by the method of claim 40;
receive measurement data comprising an identification of concentrations of metabolites in a sample of a microbiome of the animal subject;
filter the measurement data for concentrations of the set of predictor metabolites in the sample; and
provide an output signal which is indicative of one or more of the concentrations of respective predictor metabolites corresponding to or deviating from one or more reference concentration for the respective predictor metabolites.
